(19) United States
(12) Patent Application Publication
(10) Pub. No.: US 2006/0173542 A1
(43) Pub. Date: Aug. 3, 2006

(54) Biomaterial for artificial cartilage

(75) Inventor: Yasuo Shikinami, Osaka-shi (JP)

Correspondence Address: SUGHRUE MION, PLLC, 2100 PENNSYLVANIA AVENUE, N.W., SUITE 800, WASHINGTON, DC, 20037, US

(73) Assignee: TAKIRON CO., LTD.

(21) Appl. No.: 11/316,704

(22) Filed: Dec. 27, 2005

Related U.S. Application Data
(60) Provisional application No. 60/639,401, filed on Dec. 28, 2004.

(51) Int. Cl. A61F 2/30; A61F 2/44

(52) U.S. Cl. 623/14.12; 623/17.16

(57) ABSTRACT

A biomedical material for artificial cartilage is provided which employs a core material comprising a structure made of organic fibers, is flexible and has nearly ideal deformation properties, can be bonded and fixed to living-body bones such as vertebral bodies without fail at a high force, and is free from the generation of fine particles caused by wearing. The biomedical material for artificial cartilage comprises a core material comprising a structure which is either a three-dimensional woven structure or knit structure made of organic fibers arranged along three or more axes or a structure comprising a combination of the woven structure and the knit structure and plates superposed respectively on the upper and lower sides of the core material, the plates being made of a biodegradable and bioabsorbable polymer containing bioactive bioceramic particles.

### BACKGROUND OF THE INVENTION

The present invention relates to a biomedical material for artificial cartilage which is expected to be used as an artificial intervertebral disk or artificial meniscus or as various articular cartilages or the like.

Metallic and ceramic materials have hitherto been used as implantation materials to be implanted in the living body. However, since these implantation materials are rigid and difficult to deform, it is difficult to use them as biomaterials for cartilages such as, e.g., intervertebral disks.

The stand-alone artificial intervertebral disks of the whole replacement type which are presently in clinical trial use although their functions are insufficient comprise the following common components and have the following common structure. Namely, the artificial intervertebral disks are artificial intervertebral disks of the so-called sandwich structure comprising a core made of bioinert polyethylene or a rubber having biocompatibility and, superposed on each of the upper and lower sides thereof, a metallic end plate made of titanium or cobalt-chromium. In the case where the core part is constituted of two sheets of polyethylene, the artificial intervertebral disk moves like intervertebral disks of the living body based on changes in the degree of superposition of the polyethylene sheets. In the case where the core part is a rubber, this core part moves like intervertebral disks of the living body due to its elasticity. Some upper and lower metallic plates have been surface-treated with hydroxyapatite so as to have an improved affinity for (bondability to) bones. For the purposes of preventing falling off after insertion between vertebral bodies and imparting a stand-alone effect, the artificial intervertebral disks have a structure in which the metallic plates each have several horns protruding from a surface thereof so that these horns stick into the surface of a vertebral body and thereby fix the artificial intervertebral disk. However, these artificial intervertebral disks have the following drawbacks which may be fatal.

a) First, since the sandwich structure comprises different materials, i.e., metallic plates and either a plastic (rigid polyethylene plates) or a rubber, this type of artificial intervertebral disk undergo wearing at the interfaces between the two kinds of materials when the artificial intervertebral disk moves repeatedly under the sandwiching pressure of vertebral bodies. This phenomenon is significant when the artificial intervertebral disk is not correctly inserted and disposed.

(b) The movement of the artificial intervertebral disks is never equal to that of intervertebral disks of the living body and inhibits natural movements.

(c) The horns protruding from the metallic plates damage the upper and lower vertebral bodies and, simultaneously, there is a considerable possibility that the horns might gradually penetrate into the vertebral bodies during long-term use to newly cause a disorder.

(d) The artificial intervertebral disk may fall off or break itself during long-term use, and there is a strong fear that the falling off or breakage may generate small pieces which cause damage to surrounding tissues or nerves.

Besides the artificial intervertebral disks described above, there is an all-metallic artificial intervertebral disk which has springs inside as a substitute for a core. However, this all-metallic artificial intervertebral disk is not thought to be usable as a substitute for an intervertebral disk of the living body with respect to any of the material, constitution, movement, and durability (corrosion resistance) thereof.

The present applicant hence proposed a biomaterial for use as an artificial cartilage such as, e.g., a stand-alone type artificial intervertebral disk (see JP-A-2003-230583). This biomaterial comprises: a core material comprising a fibrous structure which is either a three-dimensional woven structure or knit structure made of organic fibers arranged along three or more axes or a structure comprising a combination of these; spacers which have been superposed respectively on both sides of the core material and which have interconnected pores and comprise a porous object of a biodegradable and bioabsorbable polymer containing bioactive bioceramic particles; and biodegradable and bioabsorbable pins for fixing which have been disposed so that the tips of each pin slightly protrude from the spacer surfaces.

When this biomedical material for artificial cartilage is inserted as an artificial intervertebral disk between adjacent vertebral bodies, the tips of each fixing pin which protrude from the spacer surfaces slightly bite into the terminal plates of the vertebral bodies to thereby fix the biomaterial between the vertebral bodies and prevent it from suffering positional shifting/falling off. In addition, the core material comprising the fibrous structure has almost the same mechanical flexibility (movability) as intervertebral disks of the living body and the deformation properties thereof are highly biomimetic. Furthermore, the spacers superposed directly bond to the upper and lower vertebral bodies and are replaced by bone tissues with the lapse of time to thereby fix the surfaces of the core material to the upper and lower vertebral bodies. Because of these, the biomedical material for artificial cartilage can effectively function as a substitute for an intervertebral disk of the living body.

The biomedical material for artificial cartilage described above is exceedingly effective in bonding to vertebral bodies because the spacers have excellent bone conductivity or bone inductivity. However, there is a fear that the spacers may deform due to compression by load with the penetration of bone tissues into the spacers and the growth thereof. There has hence been a possibility that the replacement of the spacers by bone tissues and the bonding between vertebral bones and the biomedical material for artificial cartilage might remain incomplete in a short period after implantation, resulting in a lowered force of bonding/fixing to the upper and lower vertebral bodies. Furthermore, the spacers comprising a porous object are brittle and, hence, there also has been a possibility that the peripheries of the spacers wear to generate fine particles.

### SUMMARY OF THE INVENTION

The invention has been achieved under the circumstances described above. An object of the invention is to provide a biomedical material for artificial cartilage which employs a core material comprising a structure made of organic fibers, is flexible and has nearly ideal deformation properties, can be bonded and fixed to vertebral bodies without fail at a high force, and is free from the generation of fine particles caused by wearing.

In order to accomplish the object, the biomedical material for artificial cartilage of the invention comprises a core material comprising a structure which is either a three-dimensional woven structure or knit structure made of organic fibers arranged along three or more axes or a structure comprising a combination of the woven structure and the knit structure and plates superposed respectively on the upper and lower sides of the core material, the plates being made of a biodegradable and bioabsorbable polymer containing bioactive bioceramic particles.

When the biomedical material for artificial cartilage of the invention is inserted, for example, as an artificial intervertebral disk between cervical or vertebral (especially lumbar vertebral) bodies, the biomaterial of the invention sufficiently functions as an intervertebral disk because the core material, which comprises a structure which is either a three-dimensional woven structure or knit structure made of organic fibers arranged along three or more axes or a structure comprising a combination of the woven structure and the knit structure, has almost the same mechanical strength and flexibility as intervertebral disks, which are cartilages, and the deformation properties thereof are highly biomimetic. In addition, since the plates superposed on the core material are plates made of a biodegradable and bioabsorbable polymer containing bioceramic particles, hydrolysis and absorption proceed from the plate surfaces upon contact with a body fluid. With this degradation/absorption, bone tissues grow conductively toward inner parts of the plates due to the bone conductivity of the bioceramic particles. In this stage, the nonporous plates made of a biodegradable and bioabsorbable polymer have a lower rate of degradation/absorption than the spacers comprising a porous object and the degradation/absorption rate thereof is substantially balanced with the rate of growth of bone tissues. Because of this, the plates gradually disappear with the degradation/absorption thereof. Simultaneously therewith, bone tissues grow and directly bond to the plates. Thereafter, the plates are further degraded and absorbed and, finally, the plates are completely replaced by bone tissues and the core material directly bonds to the vertebral bodies. Thus, the force of bonding and fixing to the vertebral bodies can be secured. In addition, since the plates made of a biodegradable and bioabsorbable polymer are not brittle, the plates can be prevented from generating fine particles even when the artificial intervertebral disk repeatedly undergoes biomimetic deformations under the high sandwiching pressure of the upper and lower vertebral bodies.

In the artificial cartilage material of the invention, the plates each may be a forged material of a biodegradable and bioabsorbable polymer containing bioactive bioceramic particles. Many perforations may be formed in the plates so as to result in a perforation rate of 15-60%. Furthermore, the perforations may be partly or wholly filled with a biodegradable and bioabsorbable material having higher bone conductivity and/or bone inductivity than the plates and showing biodegradation at a higher rate than the plates. Moreover, a covering layer made of a biodegradable and bioabsorbable material having higher bone conductivity and/or bone inductivity than the plates and showing biodegradation at a higher rate than the plates may be formed on the obverse side of each plate or on each of the obverse and reverse sides thereof.

The biodegradable and bioabsorbable material to be packed into the perforations of the plates and the biodegradable and bioabsorbable material constituting the covering layer to be superposed on the plates preferably are: one which is a porous object of a biodegradable and bioabsorbable polymer, has interconnective pores, and contains bioceramic particles having bone conductivity and/or one or more of a cytokine having bone inductivity, a drug having bone inductivity, and a bone inductive biological factor; or one which comprises collagen and, incorporated therein, bioceramic particles having bone conductivity and/or one or more of a cytokine having bone inductivity, a drug having bone inductivity, and a bone inductive biological factor.

Furthermore, in the biomedical material for artificial cartilage of the invention, fine concave and convex surface may be formed on each of the obverse and reverse sides of each plate, and the periphery of each plate may be sewed to the core material with a yarn. It is also possible to dispose at least one biodegradable and bioabsorbable pin so that the pin extends through the core material and the plates and the tips of the pin protrude from the plate surfaces.

### BRIEF DESCRIPTION OF THE DRAWINGS

FIG. 1 is a slant view illustrating one embodiment of the biomedical material for artificial cartilage of the invention.

FIG. 2 is a sectional view taken on the line A-A of FIG. 1.

FIG. 3 is a view illustrating an example of the use of a biomedical material for artificial cartilage of the invention.

FIG. 4 is a sectional view illustrating another example of the plates for use in the biomedical material for artificial cartilage of the invention.

FIG. 5 is a slant view illustrating another embodiment of the biomedical material for artificial cartilage of the invention.

FIG. 6 is a sectional view taken on the line B-B of FIG. 5.

FIG. 7 is a sectional view illustrating still another example of the plates for use in the biomedical material for artificial cartilage of the invention.

FIG. 8 is a sectional view illustrating a further example of the plates for use in the biomedical material for artificial cartilage of the invention.

FIG. 9 is a slant view illustrating still another embodiment of the biomedical material for artificial cartilage of the invention.

FIG. 10 is a sectional view taken on the line C-C of FIG. 9.

FIG. 11 is a sectional view illustrating a further embodiment of the biomedical material for artificial cartilage of the invention.

FIG. 12 is a slant view illustrating still a further embodiment of the biomedical material for artificial cartilage of the invention.

FIG. 13 is a slant view illustrating still a further embodiment of the biomedical material for artificial cartilage of the invention.

FIG. 14 is a plan view showing insertion positions of biomaterials for artificial cartilages of the invention.

FIG. 15 is a slant view illustrating still a further embodiment of the biomedical material for artificial cartilage of the invention.

FIG. 16 is a slant view illustrating still a further embodiment of the biomedical material for artificial cartilage of the invention.

### DETAILED DESCRIPTION OF THE INVENTION

Embodiments of the invention will be explained below by reference to the drawings.

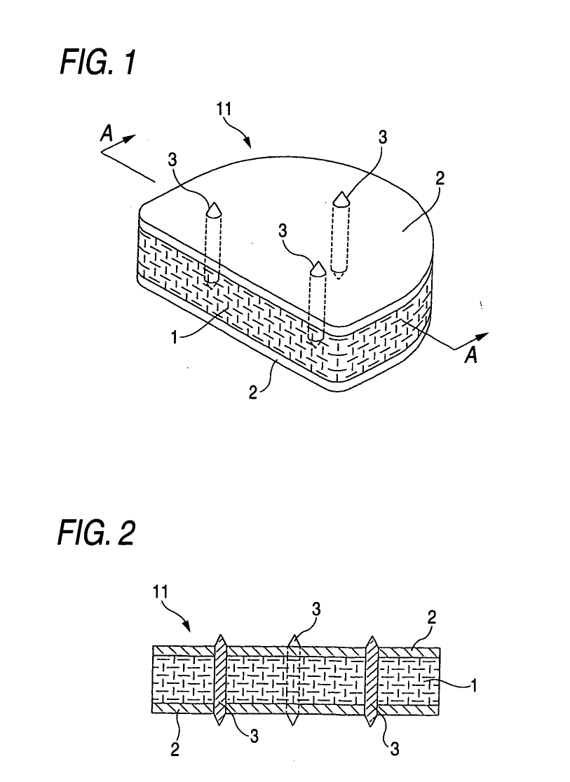

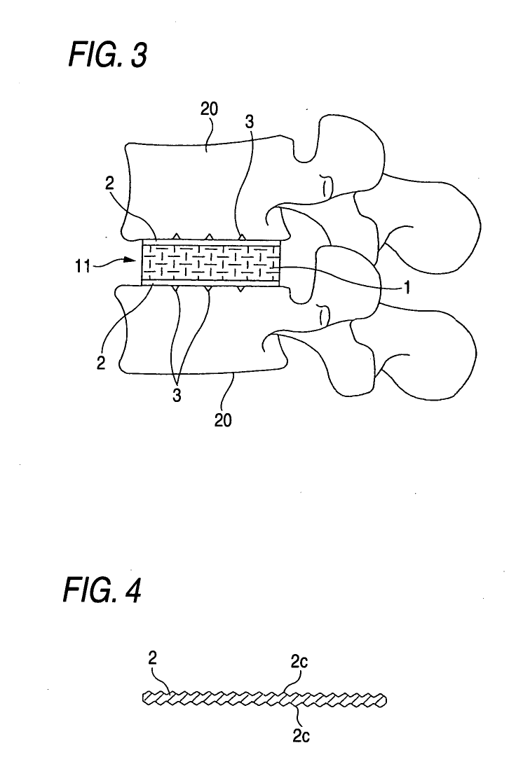

The biomedical material for artificial cartilage 11 shown in FIG. 1 is in a block form having a planar shape which is nearly square at the front and rounded at the rear, i.e., which comprises a rectangular front-half part and a semi-circular rear-half part united therewith. This biomaterial 11 is intended to be inserted as a whole replacement type artificial intervertebral disk between adjacent vertebral bodies 20 and 20 in the vertebral (especially lumbar vertebral) column or the cervical vertebral column from the obverse side as shown in FIG. 3. This size of this biomedical material for artificial cartilage 11 varies depending on whether it is for use as an artificial intervertebral disk for the cervical vertebrae or as an artificial intervertebral disk for lumbar vertebrae and depending on whether it is for adults or for children. For example, in the case where the biomaterial 11 is for use as an artificial intervertebral disk for cervical vertebrae of adults, it normally has a width dimension of about 18 mm, length dimension of about 15 mm, and thickness dimension of about 7 mm. In the case where the biomaterial 11 is for use as an artificial intervertebral disk for lumbar vertebrae of adults, it normally has a width dimension of about 40 mm, length dimension of about 30 mm, and thickness dimension of about 15 mm.

As shown in FIG. 1 and FIG. 2, this biomedical material for artificial cartilage 11 comprises a core material 1 and plates 2 and 2 superposed respectively on the upper and lower sides thereof and has three biodegradable and bioabsorbable pins 3 extending through the core 1 and the plates 2 and 2, the tips of each pin 3 slightly protruding from the surfaces of the plates 2 and 2.

The core material 1 comprises a structure which is either a three-dimensional woven structure or knit structure made of organic fibers or a structure comprising a combination of the woven structure and the knit structure. It is a core material having almost the same mechanical strength and flexibility as cartilages, such as intervertebral disks, of the living body and the deformation properties thereof are highly biomimetic. The structure of this core material 1 is the same as the structure described in Japanese Patent Application No. 1994-254515 (JP-A-7-148243), which was filed by the applicant. When the geometry of this core material is expressed in terms of the number of dimensions and the number of directions of fiber arrangement is expressed in terms of the number of axes, then the structure preferably is a three-dimensional structure with three or more axes.

The three-axis three-dimensional structure is a structure made up of three-dimensionally arranged fibers extending in three axial directions, i.e., length, width, and vertical directions. A typical shape of this structure is a thick bulk shape (platy or block shape) such as the core material 1. However, a cylindrical or honeycomb shape is also possible. This kind of three-axis three-dimensional structures are classified, according to structure differences, into orthogonal structure, non-orthogonal structure, leno structure, cylindrical structure, etc. A three-dimensional structure with four or more axes has an advantage that the strength isotropy of the structure can be improved by arranging fibers in directions along 4, 5, 6, 7, 9, or 11 axes, etc. By selecting these, a core material 1 which is more biomimetic and more akin to cartilage tissues of the living body can be obtained.

The core material 1 comprising the structure described above preferably has an internal porosity in the range of 20-90%. In case where the internal porosity thereof is lower than 20%, this core material 1 is too dense and is impaired in flexibility and deformability. This material is hence unsatisfactory as the core material of a biomedical material for artificial cartilage. In case where the internal porosity thereof exceeds 90%, this core material 1 is reduced in compression strength and shape retention. This material also is hence unsuitable for use as the core material of a biomedical material for artificial cartilage.

As the organic fibers which constitute the core material 1 are preferably used bioinert synthetic resin fibers such as, e.g., fibers of polyethylene, polypropylene, polytetrafluoroethylene, or the like and coated fibers obtained by coating organic core fibers with any of these bioinert resins to impart bioinertness. In particular, coated fibers having a diameter of about 0.2-0.5 mm obtained by coating core fibers of ultrahigh-molecular polyethylene with linear low-density polyethylene are optimal fibers from the standpoints of strength, hardness, elasticity, suitability for weaving/knitting, etc. Besides these, fibers having bioactivity (e.g., having bone conductivity or bone inductivity) can be selected.

A further explanation on the structure which constitutes the core material 1 is omitted because the structure is disclosed in detail in Japanese Patent Application No. 1994-254515 (JP-A-7-148243), which was cited above.

The plates 2 and 2 superposed respectively on the upper and lower sides of the core material 1 are nonporous plates made of a biodegradable and bioabsorbable polymer containing bioactive bioceramic particles. Use may be made of one obtained by melt-molding the polymer or one obtained by subjecting the melt-molded object to cold forging (at a temperature which is higher than the glass transition temperature of the polymer and is lower than the melting temperature thereof).

The latter plates, i.e., forged plates, may be ones obtained by forging the melt-molded object once or may be ones obtained by forging it two or more times. In particular, however, plates obtained by subjecting an object which was forged once to forging once again in a changed machine direction have an advantage that they are less apt to deteriorate mechanically or break even when repeatedly deformed by external forces, because the thus-forged plates have a dense structure in which molecular chains or crystal axes of the polymer have been oriented along many reference axes randomly different in three-dimensional directions, or a structure made up of many clusters of these which have many reference axes randomly different, or a dense structure in which molecular chains, crystals, and clusters are oriented in three-dimensional directions. Consequently, when a biomedical material for artificial cartilage 11 comprising a core material 1 and, superposed on each side thereof, such a plate 2 which has under gone forging twice is inserted between vertebral bodies 20 and 20, then the plates 2 do not suffer mechanical deterioration, breakage, or the like until the plates 2 are mostly degraded and absorbed, even when the plates 2 are repeatedly deformed together with the core material 1 by the sandwiching pressure of the upper and lower vertebral bodies 20 and 20. Furthermore, even the plates which have undergone forging once have improved mechanical strength and less susceptibility to breakage as compared with plates obtained through mere melt molding, because the plates have been densified by compression and come to have a three-dimensionally oriented structure in which molecular chains or crystals of the polymer are oriented obliquely to one reference axis or reference plane or a three-dimensionally oriented structure in which the molecular chains or crystals are oriented along many axes as described above.

Preferred examples of the biodegradable and bioabsorbable polymer to be used as a material of the plates 2 include poly(lactic acid)s, such as poly(L-lactic acid), poly(D-lactic acid), and poly(D, L-lactic acid), and copolymers of any of L-lactide, D-lactide, and DL-lactide with glycolide, caprolactone, dioxanone, ethylene oxide, or propylene oxide. These may be used alone or as a mixture of two or more thereof. Of these polymers, the poly(lactic acid)s preferably are ones having a viscosity-average molecular weight of about 50,000-500,000 from the standpoints of the rate and period (1-odd year) of degradation/absorption of the plates 2 which are balanced with the growth of bone tissues and the mechanical strength which enables the plates 2 to withstand the sandwiching pressure of vertebral bodies, etc.

As the bioceramic particles to be incorporated in the biodegradable and bioabsorbable polymer, use is made of ones having bioactivity and having satisfactory bone conductivity and satisfactory biocompatibility, such as uncalcined or unburned particles of hydroxyapatite, dicalcium phosphate, tricalcium phosphate, tetracalcium phosphate, octacalcium phosphate, calcite, Ceravital, diopside, or natural coral. Also usable are ones obtained by adhering an alkaline inorganic compound or a basic organic substance to the surface of these particles. Preferred of these are in vivo wholly absorbable bioceramic particles which are wholly absorbed in the living body and completely replaced by bone tissues. In particular, uncalcined or unburned hydroxyapatite, tricalcium phosphate, and octacalcium phosphate are optimal because they have exceedingly high activity and excellent bone conductivity, are less harmful, and are absorbed by the living body in a short period. The particles of any of these bioceramics to be used have an average particle diameter of 10 μm or smaller, preferably about 0.2-5 μm.

The content of the bioceramic particles is preferably regulated to 20-60% by mass. Contents thereof exceeding 60% by mass are disadvantageous because the plates 2 become brittle and are hence apt to break due to the sandwiching pressure of vertebral bodies. Contents thereof lower than 20% by mass are disadvantageous because the conductive growth of bone tissues becomes slow and, hence, a prolonged period is required for the plates 2 to be replaced by bone tissues. The content of the bioceramic is more preferably 25-50% by mass.

Besides the bioceramic particles, various cytokines having bone inductivity and drugs having bone inductivity may be incorporated into the plates 2 in a suitable amount. In this case, there is an advantage that the growth of and replacement by bone tissues, which occur with the degradation/absorption of the plates 2, are considerably accelerated and the core material 1 is directly bonded to vertebral bodies 20 in an early stage. A bone inductive biological factor (bone morphogenetic protein) may also be incorporated into the plates 2. This incorporation is effective in further enhancing bonding/integration because bone induction occurs. Drugs having various effects (remedial agents, etc.) may be incorporated into the plates 2 according to need. Furthermore, both sides of each plate 2 may be subjected to an oxidation treatment such as corona discharge, plasma treatment, or hydrogen peroxide treatment. In this case, the wettability of the bioceramic particles exposed on the surfaces is improved and the penetration and growth of bone cells to be proliferated come to occur effectively.

The bioceramic particles, cytokine, drug, bone inductive biological factor, etc. maybe applied by spraying to the surfaces of the core material 1. In this case, there is an advantage that since the surfaces of the core material 1 become bioactive and bone tissues which have conductively grown bond to the activated surfaces, direct bonding between vertebral bodies 20 and the core material 1 is accomplished in a relatively short period while maintaining strength.

It is preferred that fine concave and convex surface be formed on each of the obverse and reverse sides of each plate 2 as shown in FIG. 4. Such concave and convex surface bring about the following advantages. When the biomedical material for artificial cartilage 11 is inserted as an artificial intervertebral disk between vertebral bodies 20 and 20, the protrusions 2c of the concave and convex surface formed on the obverse side of each plate 2 bite into the terminal plate of the vertebral body 20 to prevent the biomedical material for artificial cartilage 11 from suffering positional shifting/falling off. Furthermore, the concave and convex surface on the obverse side are effective in bonding because they considerably increase the area of contact with the vertebral body 20. On the other hand, the protrusions 2c of the concave and convex surface formed on the reverse side of each plate 2 bite into the core material 1 to prevent the plate 2 and the core material 1 from suffering relative positional shifting. Consequently, in the case where such concave and convex surface are formed on both sides of each plate 2, the pins 3 may be omitted.

The fine concave and convex surface may have random shapes. However, the concave and convex surface preferably are ones in which the protrusions 2c are many fine protrusions of a pyramid shape (e.g., a regular quadrangular pyramid shape in which each side of the square bottom face has a length of about 0.6 mm and the pyramid height is about 0.3 mm) arranged closely so that each protrusion is not spaced from the adjacent ones. The formation of such concave and convex surface has an advantage that since the pyramidal protrusions 2c are apt to bite into the terminal plate of the vertebral body 20 and into the core material 1, the positional shifting/falling off of the biomedical material for artificial cartilage 11 and the relative positional shifting of each plate 2 and the core material 1 can be prevented with higher certainty.

The thickness of each plate 2 is desirably regulated to a value in the range of 0.3-1.2 mm, especially preferably to about 1 mm. In the case where fine concave and convex surface are formed on both sides of each plate 2, it is preferred that the thickness of the thinnest parts (the distance between the recess bottom on one side and the recess bottom on the other side) be regulated to 0.3 mm or larger and the thickness of the thickest parts (the distance between the top of the protrusion 2c on one side and the top of the protrusion 2c on the other side) be regulated to 1.2 mm or smaller. The plates 2 having such specific values of thickness have advantages that they have a strength which enables the plates 2 to withstand the sandwiching pressure of the upper and lower vertebral bodies 20 and 20, and that the plates 2 are degraded and absorbed at a rate balanced with the growth of bone tissues and are completely replaced by bone tissues to complete tenacious bonding to the vertebral bodies 20 in 1-odd year. In case where the thickness of each plate 2 (thickness of the thinnest parts when concave and convex surface have been formed on both sides) is smaller than 0.3 mm, there is a possibility that the plates 2 might have insufficient strength and break due to the sandwiching pressure of the vertebral bodies 20 and 20. On the other hand, in case where the thickness of each plate 2 (thickness of the thickest parts when concave and convex surface have been formed on both sides) is larger than 1.2 mm, a trouble arises that the time period required for the degradation/absorption of the plates 2 is prolonged and replacement by bone tissues becomes slow.

The pins 3 which vertically extend through the core material 1 and the two plates 2 and 2 disposed respectively on both sides of the core material 1 preferably are pins which are made of the lactic acid polymer described above and the strength of which has been heightened by orienting polymer molecules or crystals through forging conducted once or twice or through stretching. The tips of each pin 3 which protrude from the plates 2 and 2 have been formed in a conical shape having a height of about 0.3-2 mm so that when this biomedical material for artificial cartilage 11 is inserted as an artificial intervertebral disk between vertebral bodies 20 and 20, the tips of each pin 3 deeply bite into the terminal plates of the vertebral bodies 20 and 20 to thereby prevent the positional shifting/falling off of the biomedical material for artificial cartilage 11 without fail. With respect to the thickness of the pins 3, the diameter thereof is desirably regulated to about 0.5-3 mm, preferably about 1 mm, so as to prevent the pins 3 from being broken or damaged by the sandwiching pressure of the vertebral bodies 20 and 20.

The biomedical material for artificial cartilage 11 may have only one pin 3. However, disposition of only one pin 3 has a drawback that although this biomedical material for artificial cartilage 11 may be prevented from suffering lateral-direction positional shifting, it cannot be prevented from rotating. It is therefore desirable to dispose two or more pins. Preferably, three pins extending through the biomedical material for artificial cartilage 11 are disposed in symmetrical positions with respect to right-and-left symmetry as shown in FIG. 1 and FIG. 2. This disposition of three pins 3 has an advantage that the biomedical material for artificial cartilage 11 can be stably attached to a position between the upper and lower vertebral bodies 20 and 20 due to three-point support. However, when the biomedical material for artificial cartilage 11 is a small one for use as a whole replacement type artificial intervertebral disk for cervical vertebrae, then two pins 3 suffice which extend through the biomaterial 11 respectively in the right and left parts thereof.

It is preferred that the bioceramic particles described above and any of various cytokines, drugs, bone inductive biological factors, and the like should be incorporated also into the pins 3 in a suitable amount. In some cases, the pins 3 may be united with the plates 2 and 2 by adhesive bonding, fusion bonding, etc. Furthermore, use may be made of a method in which each pin 3 is divided into an upper part and a lower part and these upper and lower pins are disposed so that the upper tip of the upper pin and the lower tip of the lower pin protrude respectively from the upper and lower plates 2 and 2.

When the biomedical material for artificial cartilage 11 having the constitution described above is inserted as an artificial intervertebral disk, for example, between adjacent lumbar vertebral bodies 20 and 20 from the obverse side, the pointed tips of each pin 3 which protrude from the obverse sides of the plates 2 and 2 of the biomedical material for artificial cartilage 11 bite into the terminal plates of the vertebral bodies 20 and 20 as shown in FIG. 3. As a result, the biomedical material for artificial cartilage 11 is sandwiched between the vertebral bodies 20 and 20 and fixed thereto without undergoing positional shifting/falling off. The core material 1, which comprises a structure made up of organic fibers and having almost the same mechanical strength and flexibility as intervertebral disks of the living body, biomimetically deforms to sufficiently function as an intervertebral disk. Even when the core material 1 thus undergoes biomimetic deformations repeatedly under the high sandwiching pressure of the upper and lower vertebral bodies 20 and 20, the plates 2 and 2 of the biomedical material for artificial cartilage 11 hardly wear to generate fine particles because they are not brittle. Especially when the plates 2 and 2 each are the forging described above, repetitions of deformations hardly result in mechanical deterioration or breakage. Upon contact with a body fluid which penetrates into the narrow spaces between each plate 2 and the vertebral body 20 and between each plate 2 and the core material 1, hydrolysis and absorption proceed from the surfaces of these plates 2 and 2. With this hydrolysis/absorption, bone tissues grow conductively toward inner parts of the plates 2 and 2 due to the bone conductivity of the bioceramic particles. Since the rate of hydrolysis/absorption of the plates 2 and 2 differs little from the rate of growth of bone tissues, the whole plates 2 and 2 are finally replaced in 1-odd year by the bone tissues which grow with the degradation/absorption of the plates 2 and 2. Thus, the core material 1 directly bonds to the vertebral bodies 20 and 20 and is tenaciously fixed. Consequently, the strength of fixing to the vertebral bodies 20 and 20 is improved.

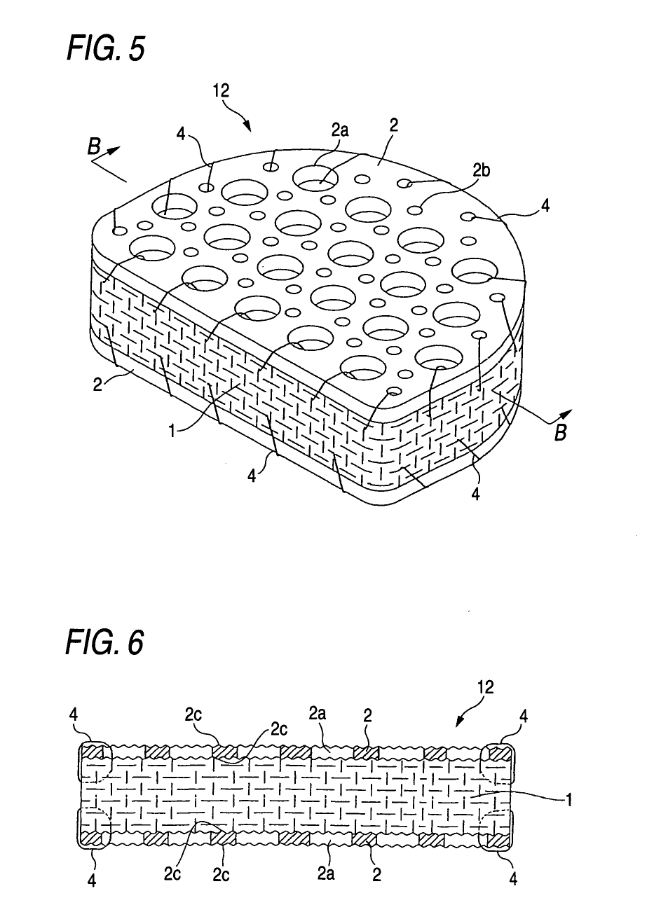

The biomedical material for artificial cartilage 12 shown in FIG. 5 and FIG. 6 comprises a core material 1 and plates 2 and 2 which each have many large perforations 2a and many small perforations 2b formed therein and which have been superposed respectively on the upper and lower sides of the core material 1. Furthermore, in this biomaterial 12, a yarn 4 has been passed through large and small perforations 2a and 2b located in peripheral parts of the plates 2 and 2 to sew the plates 2 and 2 to the core material 1 so as to cover the periphery of each of the plates 2 and 2. These plates 2 and 2 each have the fine concave and convex surface described above on each of the obverse and reverse sides thereof. By thus sewing the peripheries of the plates 2 and 2 to the core material 1 with the yarn 4, the relative positional shifting of the core material 1 and the plates 2 and 2 and the separation of the plates 2 and 2 are prevented even when the pins 3 are omitted. The yarn 4 is one comprising a bioinert fiber, biodegradable fiber, or the like. As the former fiber, i.e., bioinert one, may be used the organic fiber described above for use in constituting the core material 1. As the latter fiber, i.e., biodegradable one, may be used a fiber made of the lactic acid polymer described above. Such yarns to be used preferably are yarns (monofilaments) which have a thickness of about 0.2-3 mm and which especially preferably have been uniaxially stretched and have a high tensile strength.

The perforated plates 2 preferably are ones in which many large and small perforations 2a and 2b have been formed so that they are almost evenly dispersed and that each plate 2 come to have a perforation rate of 15-60%. The plates 2 thus regulated so as to have a perforation rate of 15-60% have a strength which enables the plates 2 to with stand the sandwiching pressure of the upper and lower vertebral bodies 20 and 20. In addition, since the perforation facilitates the penetration of a body fluid and osteoblast from the obverse side of each of the two upper and lower plates 2 and 2, bone tissues penetrate into the perforations 2a and grow between the core material land each vertebral body 20. Thus, the core material 1 directly bonds to the vertebral body 20 in the perforated parts of the plate 2 earlier than in the other parts of the plate 2. Finally, each plate 2 is wholly replaced by bone tissues and the core material 1 tenaciously bonds to the vertebral body 20. Perforation rate higher than 60% is undesirable because the plate 2 has a reduced strength. Perforation rate lower than 15% is undesirable because the effect of directly bonding the core material 1 to the vertebral body 20 through the perforations is low for use of the perforated plate.

The diameters of the large and small perforations 2a and 2b are not particularly limited. However, it is preferred to regulate the diameters of the large perforations 2a and small perforations 2b in the range of 0.5-5 mm. In case where the diameter of the large perforations 2a exceeds 5 mm, this is undesirable because the perforations 2a are less apt to be completely filled with growing bone tissues, resulting in a possibility that it might be difficult to grow bone tissues over the whole surfaces of the core material 1.

It is also possible to dispersedly form perforations having the same diameter in each plate 2, in place of forming large perforations separately from small perforations. The shape of the perforations 2a and 2b is not limited to complete circle as in this embodiment, and the perforations may be formed in any desired shape selected from ellipses, elongated circles, quadrilaterals, other polygons, irregular shapes, and the like. Consequently, quadrilateral perforations of the same size may, for example, be formed in lattice arrangement to constitute a net-form plate 2.

The core material 1 of this biomedical material for artificial cartilage 12 is the same as the core material 1 of the biomedical material for artificial cartilage 11 described above, and the plates 2 also are equal in material and others to those of the biomaterial 11. Consequently, an explanation on these is omitted.

When the biomedical material for artificial cartilage 12 described above is inserted as an artificial intervertebral disk, for example, between adjacent lumbar vertebral bodies 20 and 20 from the obverse side, the following advantage is brought about besides the same effects as those produced with the biomedical material for artificial cartilage 11 described above. Since the perforation facilitates the penetration of a body fluid and osteoblast from the obverse side of each of the two upper and lower plates 2 and 2, bone tissues penetrate into the perforations 2a and grow between the core material 1 and each vertebral body 20. Thus, the core material 1 can directly bond to the vertebral body 20 in the perforated parts of the plate 2 earlier than in the other parts of the plate 2. In addition, although this biomaterial 12 has no pins, the protrusions 2c of the fine concave and convex surface formed on the obverse side of each plate 2 bite into the terminal plate of the vertebral body 20 and, hence, the biomedical material for artificial cartilage 12 is prevented from suffering positional shifting/falling off.

In the biomedical material for artificial cartilage 12 described above, three biodegradable and bioabsorbable pins 3 of the type described above may be disposed so that they vertically extend through the biomaterial 12 and the pointed tips of each pin 3 slightly protrude from the obverse sides of the plates 2 and 2 through perforations 2a or 2b. This constitution has an advantage that the tips of each pin 3 bite into the terminal plates of the vertebral bodies 20 and 20 and the biomedical material for artificial cartilage 12 can be prevented, with higher certainty, from suffering positional shifting/falling off.

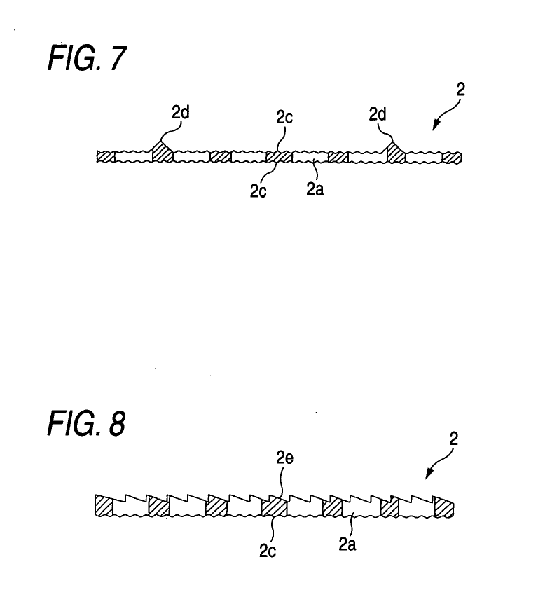

Furthermore, in the biomedical material for artificial cartilage 12 described above, the plate 2 shown in FIG. 7 or the plate 2 shown in FIG. 8 may be superposed on each of the upper and lower sides of the core material 1 in place of the plate 2 shown in FIG. 6. The plate 2 shown in FIG. 7 is the same as the perforated plate 2 shown in FIG. 6, which has fine concave and convex surface on each of the obverse and reverse sides thereof, except that this plate 2 further has pyramidal or conical projections 2d which have a larger height than those fine concave and convex surface (i.e., which have a height of 0.5-1.5 mm) formed on the obverse side thereof. A biomedical material for artificial cartilage which comprises a core material 1 and, superposed on each side thereof, this plate 2 shown in FIG. 7 has an advantage that after insertion of this biomaterial between vertebral bodies, the projections 2d deeply bite into the terminal plates of the vertebral bodies 20 and, hence, the positional shifting/falling off of the biomedical material for artificial cartilage can be prevented with higher certainty even without the pins described above. On the other hand, the plate 2 shown in FIG. 8 is the same as the perforated plate 2 shown in FIG. 6, except that this plate 2 has, on its obverse side, concave and convex surface 2e which have a saw-toothed section. A biomedical material for artificial cartilage which comprises a core material 1 and this plate 2 superposed on each side thereof so that the oblique faces of the saw-toothed concave and convex surface 2e face forward (in the direction of insertion) has an advantage that this biomaterial can be easily inserted between vertebral bodies 20 and 20 with reduced resistance and, after the insertion, does not readily fall off. In the plates 2 shown in FIG. 7 and FIG. 8, the parts equal to those in the plate 2 shown in FIG. 6 are indicated by like signs.

It is a matter of course that the pyramidal or conical projections 2d shown in FIG. 7 and the concave and convex surface 2e having a saw-toothed section shown in FIG. 8 may be formed in the plates 2 shown FIG. 1 and FIG. 2, which have no perforations and have a smooth surface on each of the obverse and reverse sides thereof, or in the plate 2 shown in FIG. 4, which has no perforations and has fine concave and convex surface formed on each of the obverse and reverse sides thereof.

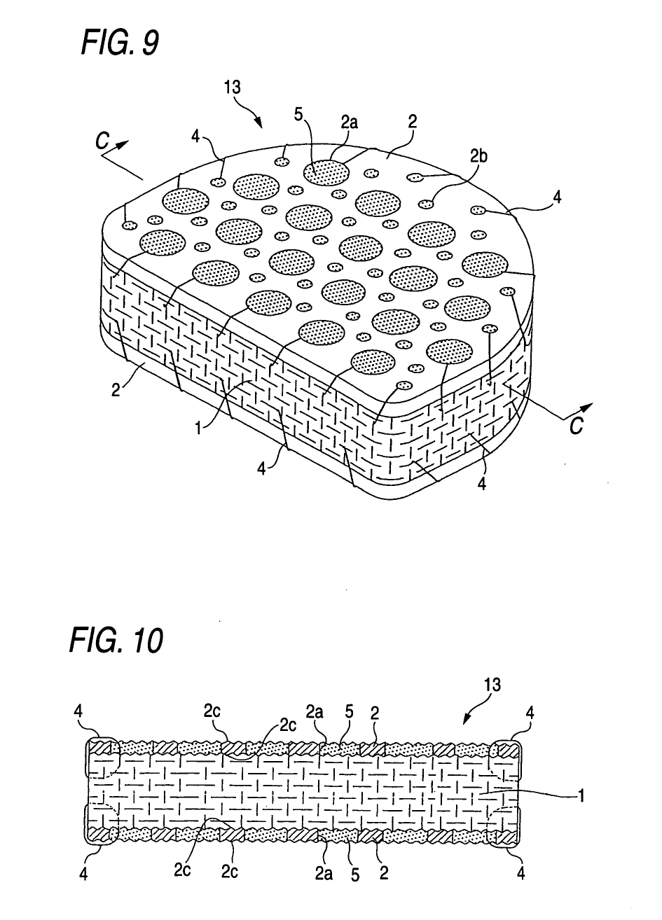

The biomedical material for artificial cartilage 13 shown in FIG. 9 and FIG. 10 is one obtained from the biomedical material for artificial cartilage 12 described above by filling the perforations 2a and 2b of each plate 2 with a biodegradable and bioabsorbable material 5 having bone conductivity and/or bone inductivity and excellent bioactivity and showing biodegradation at a higher rate than the plate 2. This biodegradable and bioabsorbable material 5 need not be always packed into all of the perforations 2a and 2b, and may be packed into part of the perforations. For example, the biodegradable and bioabsorbable material 5 may be packed into the large perforations 2a only.

The biodegradable and bioabsorbable material 5 most preferably is a porous biodegradable and bioabsorbable polymer which has interconnective pores and contains the bioceramic particles having bone conductivity and/or at least one of various cytokines having bone inductivity, drugs having bone inductivity, and bone inductive biological factors (BMF). Also preferably used is a porous or nonporous object comprising collagen and, incorporated therein, bioactive bioceramic particles and/or at least one of various cytokines having bone inductivity, drugs having bone inductivity, and bone inductive biological factors. Furthermore, a nonporous object comprising a biodegradable and bioabsorbable polymer containing bioceramic particles in a larger amount than in the plate 2 is also usable. The content of the bioceramic particles in these porous or nonporous objects is preferably regulated to 60-90% by mass. The content of the cytokine having bone inductivity, drug having bone inductivity, or bone inductive biological factor may be a suitable amount. Drugs having various effects (remedial agents, etc.) may be incorporated into the biodegradable and bioabsorbable material 5 according to need.

The porous object to be used as the biodegradable and bioabsorbable material 5 is not required to have high strength and is required to degrade more rapidly than the plates 2 and be speedily replaced by bone tissues which grow conductively and/or inductively. Because of this, a suitable biodegradable and bioabsorbable polymer for use as a raw material for this porous object is one which is amorphous or is a mixture of both crystalline state and amorphous state, and which is safe, degraded relatively rapidly, and not so brittle. Examples thereof include poly(D,L-lactic acid), copolymers of L-lactic acid and D, L-lactic acid, copolymers of a lactic acid and glycolic acid, copolymers of a lactic acid and caprolactone, copolymers of a lactic acid and ethylene glycol, and copolymers of a lactic acid and p-dioxanone. These may be used alone or as a mixture of two or more thereof. From the standpoints of the ease of porous-object formation, period of in vivo degradation/absorption, etc., these polymers to be used preferably have a viscosity-average molecular weight of about 50,000-1,000,000.

The porous object of the polymer desirably is one which has a porosity of 50-90% and in which interconnected pores account for 50-90% of all pores and the interconnected pores have a pore diameter of about 100-400 μm, when physical strength, penetration and stabilization of osteoblast, etc. are taken into account. In case where the porosity exceeds 90% and the pore diameter exceeds 400 μm, the porous object has reduced physical strength and is excessively brittle. On the other hand, when the porosity is lower than 50%, the proportion of interconnected pores is lower than 50% based on all pores, and the pore diameter thereof is smaller than 100 μm, then the penetration of a body fluid or osteoblast becomes difficult and the hydrolysis of the porous object and the growth of bone tissues become slow. In this case, the time period required for the porous object to be replaced by bone tissues is hence prolonged. A more preferred porous object is one which has a porosity of 60-80% and in which intercountered pores account for 70-90% of all pores and the interconnected pores have a pore diameter of about 150-350 μm.

Methods for producing the porous object are not particularly limited and it may be produced in any method. For example, the porous object can be produced by a method which comprises: dissolving the biodegradable and bioabsorbable polymer in a volatile solvent and mixing bioceramic particles and other ingredients therewith to prepare a suspension; forming this suspension into fibers by, e.g., spraying to obtain a fibrous mass made up of intertwined fibers; packing the fibrous mass into the perforations 2a and 2b of each plate 2 which has not been superposed; heating this plate 2 to a temperature at which the fibers are fusion-bondable to thereby partly fusion-bond the fibers to one another and obtain a porous fusion-bonded fibrous mass; and immersing this fusion-bonded fibrous mass in a volatile solvent together with the plate 2 to convert the fibrous mass into a porous object.

After the biomedical material for artificial cartilage 13 described above is inserted as an artificial intervertebral disk between adjacent vertebral bodies 20 and 20, the biodegradable and bioabsorbable material 5 with which the perforations 2a and 2b of each plate 2 are filled is degraded more rapidly than the plate 2, and bone tissues rapidly grow conductively and/or inductively due to the bone conductivity of the bioceramic particles contained in this biodegradable and bioabsorbable material 5 and the bone inductivity of the cytokine, the bone inductivity of drug, or the bone inductivity of bone inductive biological factor to replace the biodegradable and bioabsorbable material 5 in the perforations 2a and 2b in an early stage. The biomaterial 13 thus comes to bond to the vertebral bodies 20. In addition, the cytokine, the drug, or the bone inductive biological factor may be contained in the biodegradable and bioabsorbable material S.

On the other hand, each plate 2 is degraded more slowly than the biodegradable and bioabsorbable material 5 and retains sufficient strength until the biodegradable and bioabsorbable material 5 in the perforations 2a and 2b is replaced by bone tissues to some degree. Thereafter, the plates 2 are wholly replaced by bone tissues and finally attain complete and tenacious bonding to the vertebral bodies 20.

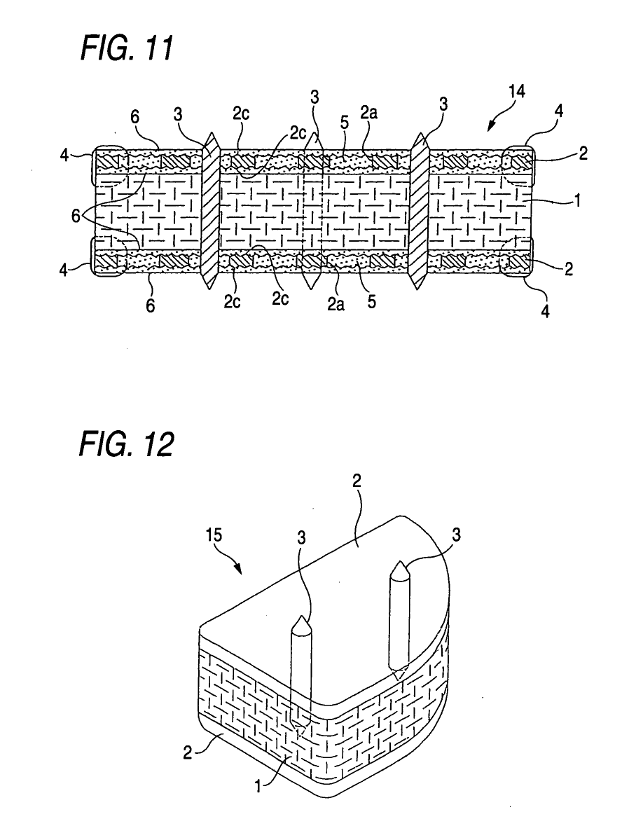

The biomedical material for artificial cartilage 14 shown in FIG. 11 is the same as the biomedical material for artificial cartilage 13 described above, except that the plates 2 and 2 each have covering layers 6 and 6 superposed respectively on the obverse and reverse sides thereof and made of a biodegradable and bioabsorbable material having bone conductivity and/or bone inductivity and that three biodegradable and bioabsorbable pins of the type described above have been disposed so that they vertically extend through the biomaterial 14 and the tips of each pin 3 slightly protrude from the obverse sides of the covering layers 6. Such a covering layer 6 need not be always superposed on each of the obverse and reverse sides of each plate 2, and may be superposed only on the obverse side of each plate 2.

The thickness of each covering layer 6 is not particularly limited. However, when the covering layer 6 is one comprising a porous object of the biodegradable and bioabsorbable polymer described above and, incorporated therein, bioceramic particles and a cytokine or the like, then the thickness thereof is preferably regulated to about 0.5-2 mm. In case where each covering layer 6 is thinner than 0.5 mm, there is a possibility that the property of coming into tight contact with a vertebral body 20 through compressive deformation is reduced. Thicknesses thereof larger than 2 mm arouse a drawback that the time period required for degradation/absorption and for replacement by bone tissues is prolonged.

When this biomedical material for artificial cartilage 14 is inserted as an artificial intervertebral disk between adjacent vertebral bodies 20 and 20, the biomedical material for artificial cartilage 14 is prevented from positional shifting/falling off by the action of the pins 3. Furthermore, with the degradation of the covering layers 6, bone tissues almost evenly grow on the surfaces of each plate 2 in an early stage to bond the plate 2 to the vertebral body 20. Especially in the case where each covering layer 6 is a porous layer comprising the biodegradable and bioabsorbable polymer described above which contains bioceramic particles and a cytokine or the like, this covering layer 6 functions as a cushioning material and comes into tight contact with a vertebral body 20 through compressive deformation to facilitate the penetration of osteoblast into inner parts of the porous layer. Consequently, bone tissues rapidly grow conductively and/or inductively, and bonding to the vertebral body 20 is accomplished in a short period.

It is a matter of course that the covering layer 6 may be superposed on each of the obverse and reverse sides of or on the obverse side of each of the plates 2 having no perforations shown in FIG. 1, FIG. 2, FIG. 4, etc.

Several examples of artificial-cartilage biomaterials for use as partial replacement type artificial intervertebral disks are shown below.

The biomedical material for artificial cartilage 15 shown in FIG. 12 is one to be used as a partial replacement type artificial intervertebral disk which replaces a half of an intervertebral disk of the vertebral (especially lumbar vertebral) column. This biomaterial 15 has a shape obtained by dividing the whole replacement type biomedical material for artificial cartilage 11 described above into a right and left part. This biomedical material for artificial cartilage 15 has the same structure as the biomedical material for artificial cartilage 11. Namely, the biomaterial 15 comprises: a core material 1 comprising a structure made up of organic fibers; plates 2 and 2 superposed respectively on the upper and lower sides of the core material 1 and made of a biodegradable and bioabsorbable polymer containing bioactive bioceramic particles; and two biodegradable and bioabsorbable pins 3 which vertically extend through the core material 1 and the plates 2 and 2 so that the tips of each pin 3 slightly protrude from the obverse sides of the plates 2 and 2.

This partial replacement type biomedical material for artificial cartilage 15 is inserted into one side of the space between vertebral bodies 20 and 20, and this insertion can be conducted from the reverse side of the lumbar vertebral column. Consequently, this biomaterial 15 can be more easily used in operations than biomaterials to be inserted between vertebral bodies from the obverse side (venter side) of the lumbar vertebral column, such as the whole replacement type biomedical material for artificial cartilage 11. Furthermore, since the core material 1 is flexible, has deformation properties akin to those of intervertebral disks of the living body, directly bonds to the vertebral bodies 20 at a high fixing force, and is free from the generation of fine particles by wearing, this biomedical material for artificial cartilage 15 is extremely suitable for use as a partial replacement type artificial intervertebral disk.

It is a matter of course that the following modifications may be made in this partial replacement type biomedical material for artificial cartilage 15: to employ plates 2 which are forgings; to form fine concave and convex surface on both sides of each plate 2; to form projections on the obverse side of each plate 2; to form perforations in each plate 2 so as to result in a perforation rate in the plate 2 of 15-60%; to fill the perforations with a biodegradable and bioabsorbable material which is degraded rapidly and has bone conductivity and/or bone inductivity; to form a covering layer made of the biodegradable and bioabsorbable material on the obverse side of or on each of the obverse and reverse sides of each plate 2; and to sew the periphery of each plate 2 to the core material with a yarn.

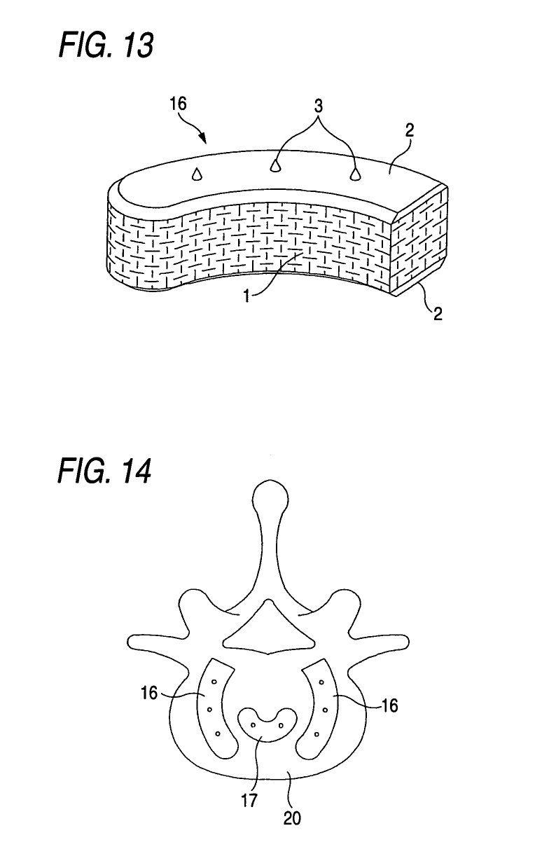

The partial replacement type biomedical material for artificial cartilage 16 shown in FIG. 13 is a biomaterial which has a circular arc shape and is rounded at one end (front end). A pair of such biomaterials 16 are inserted between vertebral (especially lumbar vertebral) bodies respectively as a right-side biomaterial and a left-side biomaterial. The size of this biomedical material for artificial cartilage 16 is normally as follows when it is for use as an artificial intervertebral disk for, e.g., lumbar vertebrae of adults. The width dimension thereof is about 9 mm and the thickness dimension thereof is about 11 mm. The radius of curvature of the circular-arc center line is about 22-23 mm, and the length dimension of the circular-arc center line is about 30 mm. Although this biomedical material for artificial cartilage 16 differs in shape from the whole replacement type biomedical material for artificial cartilage 11 described above, these biomaterials have the same structure. Namely, the biomedical material for artificial cartilage 16 comprises: a core material 1; plates 2 and 2 superposed respectively on the upper and lower sides of the core material 1; and three biodegradable and bioabsorbable pins 3 which are disposed at an interval along the center line of the biomaterial and vertically extend through the core material 1 and the plates 2 and 2 so that the tips of each pin 3 protrude from the plates 2 and 2.

A pair of such partial replacement type biomaterials for artificial cartilages 16 are inserted, respectively as a right-side biomaterial and a left-side biomaterial, between vertebral bodies 20 from the reverse side of the lumbar vertebral column as shown in FIG. 14. Since the front end of each biomedical material for artificial cartilage 16 is rounded, the front end thereof is not caught by a vertebral body and the biomaterial 16 can be smoothly inserted. The core material 1 is flexible and shows biomimetic deformations akin to those of intervertebral disks of the living body. This core material 1 directly bonds to the vertebral bodies 20 through the replacement of the plates 2 and 2 by bone tissues. Thus, the biomaterials 16 sufficiently function as an intervertebral disk.

In the case where a pair of biomaterials for artificial cartilages 16 and 16 are inserted respectively as a right-side biomaterial and a left-side material, it is preferred to insert a partial replacement type comma-shaped biomedical material for artificial cartilage 17 into the position intermediate between the biomaterials for artificial cartilages 16 and 16, as shown in FIG. 14. This biomedical material for artificial cartilage 17 also comprises a core material, two plates superposed respectively on the upper and lower sides of the core material, and two pins extending vertically through the core material and the plates so that the tips of each pin slightly protrude from the plates.

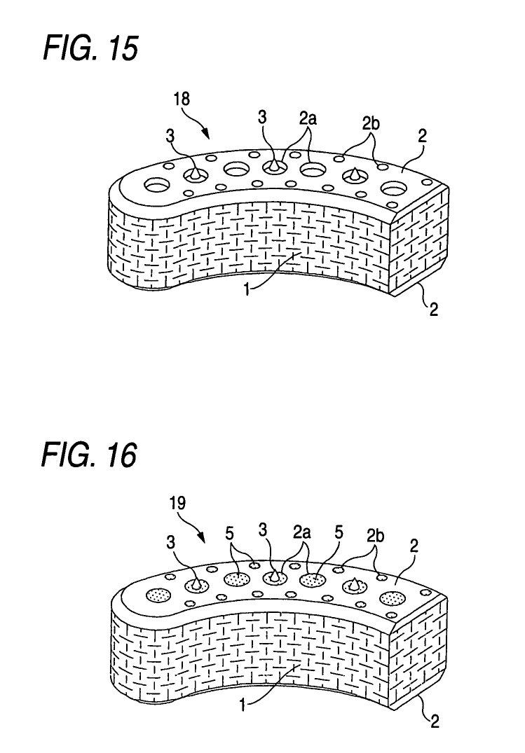

The partial replacement type biomedical material for artificial cartilage 18 shown in FIG. 15 is the same as the biomedical material for artificial cartilage 16 described above, except that large perforations 2a and small perforations 2b have been formed at an interval along the circular-arc center lines of the plates 2 and 2 and in peripheral parts of the plates 2 and 2, respectively, so as to result in a perforation rate of the plates of 15-60%, and that three pins 3 have been disposed so that they extend through some of the perforations 2a and their tips protrude. The partial replacement type biomedical material for artificial cartilage 19 shown in FIG. 16 is one obtained by filling the perforations 2a and 2b of the plates 2 and 2 of the biomedical material for artificial cartilage 18 with the biodegradable and bioabsorbable material 5 described above.

These partial replacement type biomaterials for artificial cartilages 18 and 19 also are used in the same manner as the biomedical material for artificial cartilage 16 described above. Namely, a pair of such biomaterials are inserted respectively as a right-side biomaterial and a left-side biomaterial from the reverse side of the lumbar vertebral column. The biomaterials 18 and 19 produce the same effects as the biomaterials for artificial cartilages 12 and 13 described above and sufficiently function as an intervertebral disk. Since the biomaterials 18 and 19 each are supported on three points by the three pins extending through perforations 2a formed along the circular-arc center line, the biomaterials for artificial cartilages 18 and 19 further have improved disposition stability.

It is a matter of course that the following modifications may be made in the partial replacement type biomaterials for artificial cartilages 17, 18, and 19 described above: to employ plates 2 which are forgings; to form fine concave and convex surface on both sides of each plate 2; to form projections on the obverse side of each plate 2; to form a covering layer made of the biodegradable and bioabsorbable material on the obverse side of or on each of the obverse and reverse sides of each plate 2; and to sew the periphery of each plate 2 to the core material with a yarn.

The invention was explained above with respect to typical embodiments of the biomedical material for artificial cartilage which is used as an artificial intervertebral disk of the whole replacement type and as an artificial intervertebral disk of the partial replacement type. However, it is a matter of course that the shape and size of the biomedical material for artificial cartilage of the invention can be suitably changed according to insertion positions. Furthermore, by changing the shape and size of the biomedical material for artificial cartilage of the invention to a shape similar to that of meniscus or any of various articular cartilages other than intervertebral disks, the biomaterial can, of course, be made usable as an artificial meniscus or as any of various artificial articular cartilages.

What is claimed is:

1. A biomedical material for artificial cartilage, which comprises:
a core material comprising a structure which is either a three-dimensional woven structure or knit structure made of organic fibers arranged along three or more axes, or a structure comprising a combination of the woven structure and the knit structure, and
plates superposed respectively on the upper and lower sides of the core material, wherein the plates are made of a biodegradable and bioabsorbable polymer containing bioactive bioceramic particles.

2. The biomedical material for artificial cartilage according to claim 1, wherein the plates each is a forged material of a biodegradable and bioabsorbable polymer containing bioactive bioceramic particles.

3. The biomedical material for artificial cartilage according to claim 1, wherein the plates have many perforations therein.

4. The biomedical material for artificial cartilage according to claim 3, wherein the plates have a perforation rate of 15-60%.

5. The biomedical material for artificial cartilage according to claim 3, wherein the perforations of the plates are partly or wholly filled with a biodegradable and bioabsorbable material having bone conductivity and/or bone inductivity and showing biodegradation at a higher rate than the plate.

6. The biomedical material for artificial cartilage according to claim 5, wherein the biodegradable and bioabsorbable material is a porous object of a biodegradable and bioabsorbable polymer, the porous object having interconnective pores and containing bioceramic particles having bone conductivity and/or one or more of a cytokine having bone inductivity, a drug having bone inductivity, and a bone inductive biological factor.

7. The biomedical material for artificial cartilage according to claim 5, wherein the biodegradable and bioabsorbable material comprises collagen and, incorporated therein, bioceramic particles having bone conductivity and/or one or more of a cytokine having bone inductivity, a drug having bone inductivity, and a bone inductive biological factor.

8. The biomedical material for artificial cartilage according to claim 1, 2, or 5, wherein the plates each has a covering layer formed on the obverse side thereof or on each of the obverse and reverse sides thereof, the covering layer being made of a biodegradable and bioabsorbable material having bone conductivity and/or bone inductivity and showing biodegradation at a higher rate than the plate.

9. The biomedical material for artificial cartilage according to claim 8, wherein the biodegradable and bioabsorbable material constituting the covering layer is a porous object of a biodegradable and bioabsorbable polymer, wherein the porous object has interconnective pores and contains bioceramic particles having bone conductivity and/or one or more of a cytokine having bone inductivity, a drug having bone inductivity, and a bone inductive biological factor.

10. The biomedical material for artificial cartilage according to claim 8, wherein the biodegradable and bioabsorbable material constituting the covering layer comprises collagen and, incorporated therein, bioceramic particles having bone conductivity and/or one or more of a cytokine having bone inductivity, a drug having bone inductivity, and a bone inductive biological factor.

11. The biomedical material for artificial cartilage according to claim 1, 2, 3, or 5, wherein the plates each has fine concave and convex surface formed on each of the obverse and reverse sides thereof.

12. The biomedical material for artificial cartilage according to claim 3 or 5, wherein the periphery of each plate has been sewed to the core material with a yarn.

13. The biomedical material for artificial cartilage according to claim 1, which has at least one biodegradable and bioabsorbable pin extending through the core material and the plates, the tips of the pin protruding from the plate surfaces.
